(19) United States
(12) Patent Application Publication
(10) Pub. No.: US 2018/0210041 A1
(43) Pub. Date: Jul. 26, 2018

(54) SENSOR AND ELECTRONIC DEVICE

(71) Applicant: Kabushiki Kaisha Toshiba, Tokyo (JP)

(72) Inventors: Shotaro Baba, Kawasaki (JP); Yoshihiko Fuji, Kawasaki (JP); Kei Masunishi, Kawasaki (JP); Michiko Hara, Yokohama (JP); Akiko Yuzawa, Kawasaki (JP); Shiori Kaji, Kawasaki (JP); Tomohiko Nagata, Yokohama (JP); Yoshihiro Higashi, Komatsu (JP); Kenji Otsu, Yokohama (JP); Kazuaki Okamoto, Yokohama (JP)

(73) Assignee: Kabushiki Kaisha Toshiba, Tokyo (JP)

(21) Appl. No.: 15/689,221

(22) Filed: Aug. 29, 2017

(30) Foreign Application Priority Data
Jan. 20, 2017 (JP) 2017-008321

(51) Int. Cl. G01R 33/09; G01B 7/31; G01D 5/245; G02B 7/08; G01L 9/16

(52) U.S. Cl. CPC G01R 33/091; G01B 7/31; G01D 5/245; A61B 2562/0247; G02B 7/08; G01L 9/16; G01R 33/093

(57) ABSTRACT

According to one embodiment, a sensor includes a support body, a film portion, a first sensing element, and a structure body. The first sensing element is fixed to the film portion, and includes a first magnetic layer, a first opposing magnetic layer, and a first intermediate layer. The structure body includes a first region overlapping the support body, and a second region being continuous with the first region and overlapping the film portion. The structure body includes a first structure body layer, a first opposing structure body layer, and a first structure body intermediate layer. The first opposing structure body layer is provided between the first structure body layer and the support body and between the first structure body layer and the film portion. The first structure body intermediate layer is provided between the first structure body layer and the first opposing structure body layer.

### CROSS-REFERENCE TO RELATED APPLICATIONS

This application is based upon and claims the benefit of priority from Japanese Patent Application No. 2017-008321, filed on Jan. 20, 2017; the entire contents of which are incorporated herein by reference.

### FIELD

Embodiments described herein relate generally to a sensor and an electronic device.

### BACKGROUND

A pressure sensor that uses a magnetic layer has been proposed. For example, the pressure sensor is applied to electronic devices and the like such as a microphone, etc. Stable sensing characteristics of the pressure sensor are desirable.

### BRIEF DESCRIPTION OF THE DRAWINGS

FIG. 1A to FIG. 1E are schematic views illustrating a pressure sensor according to a first embodiment;

FIG. 2A and FIG. 2B are schematic views illustrating characteristics of the pressure sensors;

FIG. 3 is a schematic plan view illustrating another pressure sensor according to the first embodiment;

FIG. 4A to FIG. 4C are schematic views illustrating another pressure sensor according to the first embodiment;

FIG. 5 is a schematic plan view illustrating another pressure sensor according to the first embodiment;

FIG. 6 is a schematic plan view illustrating another pressure sensor according to the first embodiment;

FIG. 7 is a schematic plan view illustrating another pressure sensor according to the first embodiment;

FIG. 8 is a schematic plan view illustrating another pressure sensor according to the first embodiment;

FIG. 9 is a schematic view illustrating a portion of another pressure sensor according to the first embodiment;

FIG. 10 is a schematic perspective view illustrating a portion of the pressure sensor according to the embodiment;

FIG. 11 is a schematic perspective view illustrating a portion of another pressure sensor according to the embodiment;

FIG. 12 is a schematic perspective view illustrating a portion of another pressure sensor according to the embodiment;

FIG. 13 is a schematic perspective view illustrating a portion of another pressure sensor according to the embodiment;

FIG. 14 is a schematic perspective view illustrating a portion of another pressure sensor according to the embodiment;

FIG. 15 is a schematic perspective view illustrating a portion of another pressure sensor according to the embodiment;

FIG. 16 is a schematic perspective view illustrating a portion of another pressure sensor according to the embodiment;

FIG. 17 is a schematic view illustrating an electronic device according to a second embodiment;

FIG. 18 is a schematic cross-sectional view illustrating the microphone according to the second embodiment;

FIG. 19A and FIG. 19B are schematic views illustrating a blood pressure sensor according to a third embodiment; and

FIG. 20 is a schematic view illustrating a touch panel according to a fourth embodiment.

### DETAILED DESCRIPTION

According to one embodiment, a sensor includes a support body, a film portion supported by the support body and being deformable, a first sensing element, and a structure body. The first sensing element is fixed to the film portion. The first sensing element includes a first magnetic layer, a first opposing magnetic layer provided between the first magnetic layer and the film portion, and a first intermediate layer provided between the first magnetic layer and the first opposing magnetic layer. The structure body includes a first region overlapping the support body in a first direction, and a second region being continuous with the first region and overlapping the film portion in the first direction. The first direction is from the first opposing magnetic layer toward the first magnetic layer. The structure body includes a first structure body layer, a first opposing structure body layer, and a first structure body intermediate layer. The first structure body layer includes a material included in the first magnetic layer. The first opposing structure body layer includes a material included in the first opposing magnetic layer and is provided between the first structure body layer and the support body and between the first structure body layer and the film portion. The first structure body intermediate layer includes a material included in the first intermediate layer and is provided between the first structure body layer and the first opposing structure body layer.

According to one embodiment, a sensor includes a support body, a film portion supported by the support body and being deformable, a first sensing element, and a structure body, The first sensing element is fixed to the film portion. The first sensing element includes a first magnetic layer. The structure body includes a first region and a second region. The first region overlaps the support body. The second region is continuous with the first region and overlaps the film portion. The structure body includes a first structure body layer including a material included in the first magnetic layer.

According to another embodiment, an electronic device includes one of the sensor described above, and a housing.

Various embodiments will be described hereinafter with reference to the accompanying drawings.

The drawings are schematic and conceptual; and the relationships between the thickness and width of portions, the proportions of sizes among portions, etc., are not necessarily the same as the actual values thereof. Further, the dimensions and proportions may be illustrated differently among drawings, even for identical portions.

In the specification and drawings, components similar to those described or illustrated in a drawing thereinabove are marked with like reference numerals, and a detailed description is omitted as appropriate.

### First Embodiment

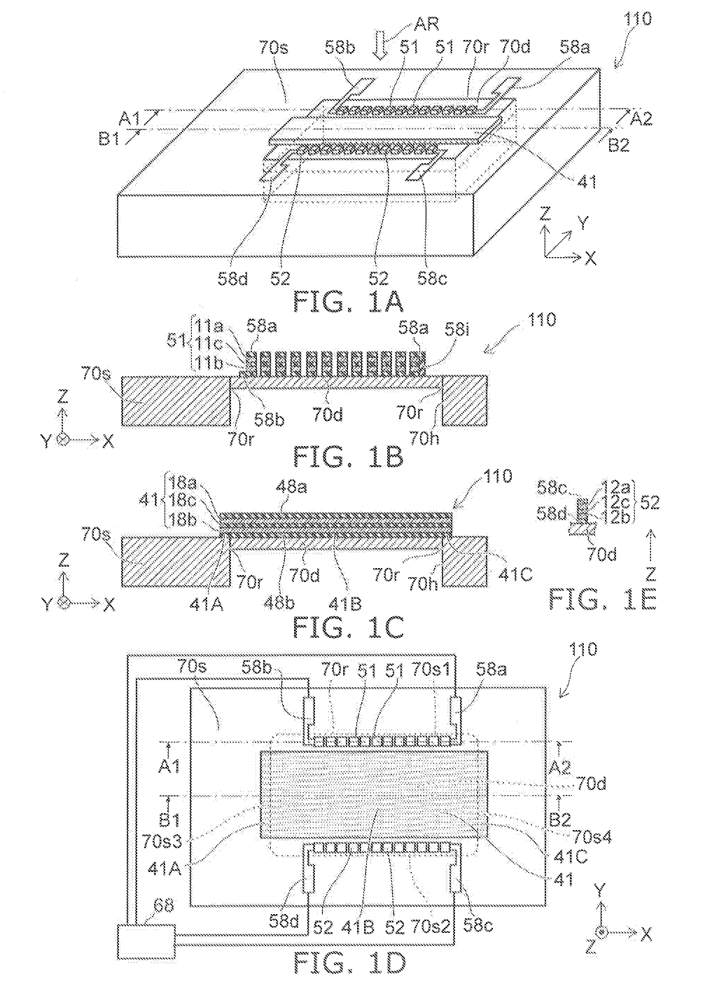

FIG. 1A to FIG. 1E are schematic views illustrating a pressure sensor according to a first embodiment. FIG. 1A is a perspective view. FIG. 1B is a line A1-A2 cross-sectional view of FIG. 1A. FIG. 1C is a line B1-B2 cross-sectional view of FIG. 1A. FIG. 1D is a plan view as viewed along arrow AR of FIG. 1A. FIG. 1E is a cross-sectional view illustrating a portion of the pressure sensor.

As shown in FIG. 1A and FIG. 1D, the pressure sensor 110 according to the embodiment includes a support body 70s, a film portion 70d, a first sensing element 51, and a structure body 41.

The film portion 70d is supported by the support body 70s. The film portion 70d is deformable. For example, the thickness of the film portion 70d is thinner than the thickness of the support body 70s. The support body 70s and the film portion 70d include, for example, silicon. For example, a recess 70h (referring to FIG. 1B and FIG. 1C) is provided in a portion of a silicon substrate. The region where the recess 70h is provided is used to form the film portion 70d. The region where the recess is not provided is used to form the support body 70s. At least a portion of an outer edge 70r of the film portion 70d is linked to the support body 70s.

The first sensing element 51 is fixed to the film portion 70d. For example, the first sensing element 51 is provided on a portion of the film portion 70d. Multiple first sensing elements 51 are provided in the example.

As shown in FIG. 1B, one of the multiple first sensing elements 51 includes a first magnetic layer 11a, a first opposing magnetic layer 11b, and a first intermediate layer 11c. The first opposing magnetic layer 11b is provided between the first magnetic layer 11a and the film portion 70d. The first intermediate layer 11c is provided between the first magnetic layer 11a and the first opposing magnetic layer 11b.

The direction from the first opposing magnetic layer 11b toward the first magnetic layer 11a is taken as a first direction. The first direction is taken as a Z-axis direction. One direction perpendicular to the Z-axis direction is taken as an X-axis direction. A direction perpendicular to the Z-axis direction and the X-axis direction is taken as a Y-axis direction. The Z-axis direction is the thickness direction of the film portion 70d.

As shown in FIG. 1C and FIG. 1D, the structure body 41 includes a first region 41A and a second region 41B. The first region 41A overlaps the support body 70s in the first direction (the Z-axis direction). The second region 41B overlaps the film portion 70d in the first direction. The second region 41B is continuous with the first region 41A. For example, a portion (the first region 41A) of the structure body 41 is provided on at least a portion of the support body 70s. Another portion (the second region 41B) of the structure body 41 is provided on a portion of the film portion 70d. The film portion 70d includes a portion where element portions such as the first sensing elements, etc., are provided, and a portion where element portions are not provided. The second region 41B is provided on the portion where element portions are not provided.

As shown in FIG. 1C, the structure body 41 includes a first structure body layer 18a, a first opposing structure body layer 18b, and a first structure body intermediate layer 18c. The first structure body layer 18a includes a material included in the first magnetic layer 11a. The first opposing structure body layer 18b is provided between the first structure body layer 18a and the support body 70s and between the first structure body layer 18a and the film portion 70d. The first opposing structure body layer 18b includes a material included in the first opposing magnetic layer 11b. The first structure body intermediate layer 18c is provided between the first structure body layer 18a and the first opposing structure body layer 18b. The first structure body intermediate layer 18c includes a material included in the first intermediate layer 11c.

For example, the structure body 41 has the same film configuration as the film configuration of the first sensing element 51. Such a structure body 41 is provided to be continuous on the support body 70s and the film portion 70d. Thereby, for example, it was found that unintended deformation of the film portion 70d can be suppressed. A pressure sensor can be provided in which the sensing characteristics can be stabilized.

A passivation film (not illustrated) may be provided on the film portion 70d and the sensing elements recited above. The characteristics of the sensing elements are stabilized by the passivation film (e.g., an insulating layer). High reliability is obtained.

For example, in a reference example in which the structure body 41 is not provided, sensing elements are not provided on the central portion of the film portion 70d. In the reference example, the surface area of the passivation film recited above contacting the film portion 70d is large. In such a case, for example, stress (residual stress) is generated between the passivation film and the film portion 70d. A large difference occurs easily in the stress between the portion of the film portion 70d in which the sensing elements are provided and the portion of the film portion 70d in which the sensing elements are not provided. Therefore, the unintended deformation of the film portion 70d occurs easily.

Conversely, in the embodiment, the structure body 41 is provided at the portion of the film portion 70d where the sensing elements are not provided. The structure body 41 has a film configuration similar to that of the sensing elements. Thereby, the difference between the stresses in the film portion 70d is suppressed. This structure body 41 is provided also on the support body 70s. Thereby, the unintended deformation of the portion (the second region 41B) of the structure body 41 on the film portion 70d is suppressed by the portion (the first region 41A) of the structure body 41 on the support body 70s. As a result, the unintended deformation of the film portion 70d is suppressed effectively. The sensing characteristics can be stabilized. An example of the suppression of the deformation is described below.

In the example as shown in FIG. 1B, the first sensing element 51 further includes a first conductive layer 58a and a second conductive layer 58b. The first magnetic layer 11a is positioned between the first conductive layer 58a and the second conductive layer 58b. The first opposing magnetic layer 11b is positioned between the second conductive layer 58b and the first magnetic layer 11a. These conductive layers function as electrodes of the first sensing element 51. In the example, an insulating layer 58i is provided between the film portion 70d and a portion of the first conductive layer 58a.

On the other hand, as shown in FIG. 1C, the structure body 41 further includes a first structure body conductive layer 48a and a second structure body conductive layer 48b. The first structure body layer 18a is positioned between the first structure body conductive layer 48a and the second structure body conductive layer 48b. The first opposing structure body layer 18b is positioned between the second structure body conductive layer 48b and the first structure body layer 18a. The first structure body conductive layer 48a includes a material included in the first conductive layer 58a. The second structure body conductive layer 48b includes a material included in the second conductive layer 58b.

For example, the first sensing element 51 and the structure body 41 are formed by forming a stacked film used to form the first sensing element 51 and the structure body 41 and by patterning the stacked film.

A second sensing element 52 is further provided in the example. The second sensing element 52 is fixed to the film portion 70d. In the example, the number of the second sensing elements 52 is multiple.

As shown in FIG. 1E, one of the multiple second sensing elements 52 includes a second magnetic layer 12a, a second opposing magnetic layer 12b, and a second intermediate layer 12c. The second opposing magnetic layer 12b is provided between the second magnetic layer 12a and the film portion 70d. The second intermediate layer 12r is provided between the second magnetic layer 12a and the second opposing magnetic layer 12b. The second sensing element 52 further includes a third conductive layer 58c and a fourth conductive layer 58d. The second magnetic layer 12a is positioned between the third conductive layer 58c and the fourth conductive layer 58d. The second opposing magnetic layer 12b is positioned between the fourth conductive layer 58d and the second magnetic layer 12a. These conductive layers function as electrodes of the second sensing element 52.

As shown in FIG. 1D, at least a portion of the second region 41B of the structure body 41 is between the first sensing element 51 and the second sensing element 52.

In the example, the outer edge 70r of the film portion 70d has a first side 70s1 and a second side 70s2. The multiple sensing elements are arranged along these sides.

The first side 70s1 extends along a second direction crossing the first direction (the Z-axis direction). The second direction is, for example, the X-axis direction. The second side 70s2 also extends along the second direction (e.g., the X-axis direction). A third direction from the second side 70s2 toward the first side 70s1 crosses a plane (e.g., the Z-X plane) including the first direction (the Z-axis direction) and the second direction (e.g., the X-axis direction). The third direction is, for example, the Y-axis direction.

The multiple first sensing elements 51 are arranged along the first side 70s1. The multiple second sensing elements 52 are arranged along the second side 70s2. A direction from one of the multiple first sensing elements 51 toward another one of the multiple first sensing elements 51 is aligned with the second direction (e.g., the X-axis direction). A direction from one of the multiple second sensing elements 52 toward another one of the multiple second sensing elements 52 is aligned with the second direction (e.g., the X-axis direction).

Such multiple first sensing elements 51 are electrically-connected in series to each other. Such multiple second sensing elements 52 are electrically-connected in series to each other. Thereby, high sensitivity is obtained.

In the example the outer edge 70r of the film portion 70d has substantially a rectangular configuration. For example, the outer edge 70r includes a third side 70s3 and a fourth side 70s4. The third side 70s3 and the fourth side 70s4 extend along the third direction (the Y-axis direction) recited above. A direction from the third side 70s3 toward the fourth side 70s4 is aligned with the second direction (e.g., the X-axis direction) recited above. The length along the second direction (e.g., the X-axis direction) of the first side 70s1 is longer than the length along the third direction (e.g., the Y-axis direction) of the third side 70s3.

The film portion 70d deforms when pressure (sound, etc.) is applied to the film portion 70d. The deformation is large in the regions along the long sides of the rectangle. By providing the sensing elements along the long sides, the pressure to be sensed can be sensed with high sensitivity.

For example, the electrical resistance of the first sensing element 51 changes according to the deformation of the film portion 70d. The change of the electrical resistance corresponds to the electrical resistance between the first conductive layer 58a and the second conductive layer 58b. The electrical resistance of the second sensing element 52 changes according to the deformation of the film portion 70d. The change of the electrical resistance corresponds to the electrical resistance between the third conductive layer 58c and the fourth conductive layer 58d.

A processor 68 (a circuit portion) is provided as shown in FIG. 1D. These electrical resistances can be sensed by the processor 68. A value (a current, a voltage, etc.) that corresponds to these electrical resistances may be sensed by the processor 68.

The change of the electrical resistance is based on, for example, the change of the orientation of the magnetization included in the magnetic layer. For example, the orientation of the magnetization of one of the first magnetic layer 11a or the first opposing magnetic layer 11b changes according to the deformation of the film portion 70d. This is based on the inverse magnetostrictive effect. Thereby, the angle between this orientation of the magnetization and the orientation of the magnetization of the other of the first magnetic layer 11a or the first opposing magnetic layer 11b changes. Thereby, the electrical resistance between the first magnetic layer 11a and the first opposing magnetic layer 11b changes. For example, this is due to the magnetoresistance effect.

The pressure sensor 110 may include the processor 68.

In the example as shown in FIG. 1D, the structure body 41 further includes a third region 41C. The third region 41C is continuous with the second region 41B. The third region 41C overlaps the support body 70s in the first direction (the Z-axis direction). In the example, at least a portion of the second region 41B is positioned between the first region 41A and the third region 41C.

Both end portions (the first region 41A and the third region 41C) of the structure body 41 are provided on the support body 70s. Thereby, the unintended deformation of the film portion 70d can be suppressed the more effectively.

For example, a portion between the first region 41A and the second region 41B overlaps the third side 70s3. For example, a portion between the third region 41C and the second region 41B overlaps the fourth side 70s4.

It is favorable for the surface area of the structure body 41 to be large. The structure body 41 covers a portion of the film portion 70d. It is favorable for the coverage of the structure body 41 to be high.

The length of the second region 41B along a direction (e.g., the Y-axis direction, etc.) crossing the first direction (the Z-axis direction) is not less than 2 times the length of the first sensing element 51 along the crossing direction (e.g., the Y-axis direction). The unintended deformation of the film portion 70d is suppressed effectively by a structure body having a large size.

The structure body 41 does not function as a sensing element. The structure body 41 is electrically insulated from the sensing elements. For example, the first structure body layer 18a is insulated from the first magnetic layer 11a and the first opposing magnetic layer 11b. The first opposing structure body layer 18b is insulated from the first magnetic layer 11a and the first opposing magnetic layer 11b. For example, the first structure body layer 18a is insulated from the second magnetic layer 12a and the second opposing magnetic layer 12b. The first opposing structure body layer 18b is insulated from the second magnetic layer 12a and the second opposing magnetic layer 12b.

The first structure body layer 18a may be electrically connected to the first opposing structure body layer 18b.

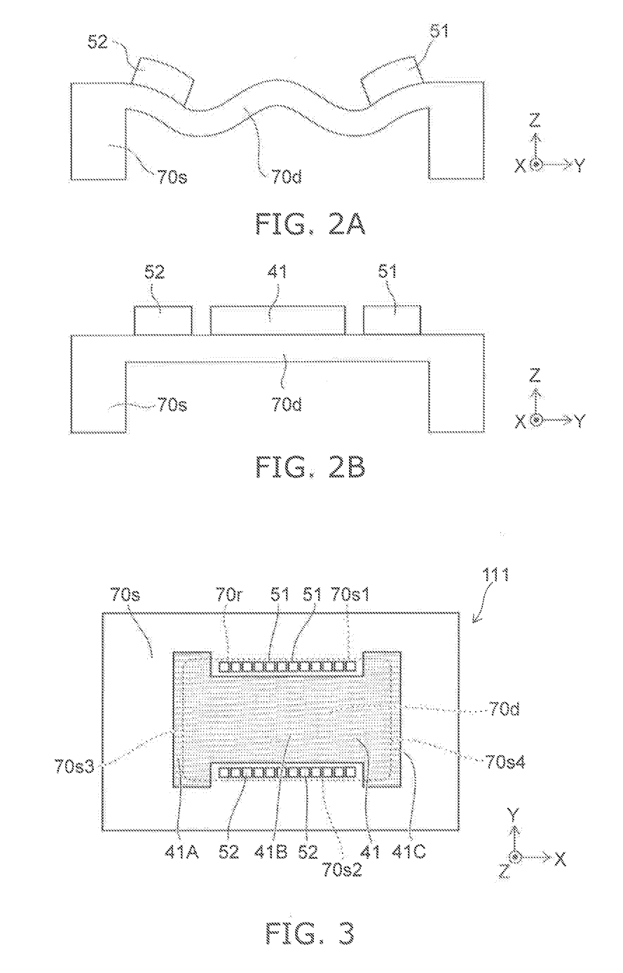

FIG. 2A and FIG. 2B are schematic views illustrating characteristics of the pressure sensors.

FIG. 2A corresponds to the reference example in which the structure body 41 is not provided. FIG. 2B corresponds to the example in which the structure body 41 is provided. These drawings correspond to cross sections when the pressure sensors are cut by the Z-Y plane. These drawings illustrate states in which the sensing object such as the sound or the like is not applied.

In the reference example as shown in FIG. 2A, the film portion 70d deforms in the downward direction at the portions where the first sensing element 51 and the second sensing element 52 are provided. The film portion 70d deforms in the upward direction at a portion between the first sensing element 51 and the second sensing element 52. The configuration of the contact with the film portion 70d is much different between the portions where the first sensing element 51 and the second sensing element 52 are provided and the portion where the first sensing element 51 and the second sensing element 52 are not provided. It is considered that this is the cause of the deformation. For example, the passivation film (not illustrated) is provided on the fifth portion 70d at the portion between the first sensing element 51 and the second sensing element 52; and it is considered that the stress generated between the passivation film and the film portion 70d is the cause of the deformation.

Conversely, as shown in FIG. 2B, the configuration of the contact with the film portion 70d is caused to be uniform in the surface of the film portion 70d by providing the structure body 41. Thereby, the stress is suppressed; and the unintended deformation of the film portion 70d can be suppressed.

As described above, the structure body 41 is provided also on the support body 70s. Thereby, the unintended deformation of the portion (the second region 41B) of the structure body 41 on the film portion 70d is suppressed by the portion (the first region 41A) of the structure body 41 on the support body 70s. As a result, the unintended deformation of the film portion 78d is suppressed effectively.

Another example of the pressure sensor according to the embodiment will now be described, in the following drawings, the first conductive layer 58a, the second conductive layer 58b, the third conductive layer 58c, the fourth conductive layer 58d, the processor 68, etc., are not illustrated.

FIG. 3 is a schematic plan view illustrating another pressure sensor according to the first embodiment.

FIG. 3 is a plan view corresponding to FIG. 1D, in the other pressure sensor 111 according to the embodiment as shown in FIG. 3, the structure body 41 includes the first region 41A, the second region 41B, and the third region 41C. In the pressure sensor 111, a portion between the first region 41A and the second region 41B overlaps the third side 70s3, a portion of the first side 70s1, and a portion of the second side 70s2. For example, the portion between the third region 41C and the second region 41B overlaps the fourth side 70s4 another portion of the first side 70s1, and another portion of the second side 70s2. Thereby, the unintended deformation of the film portion 70d can be suppressed more effectively.

For example, at least a portion of the first sensing element 51 overlaps the first region 41A in a direction (e.g., the X-axis direction) crossing the first direction (the Z-axis direction). In the example, at least a portion of the first sensing element 51 is positioned between the first region 41A and the third region 41C.

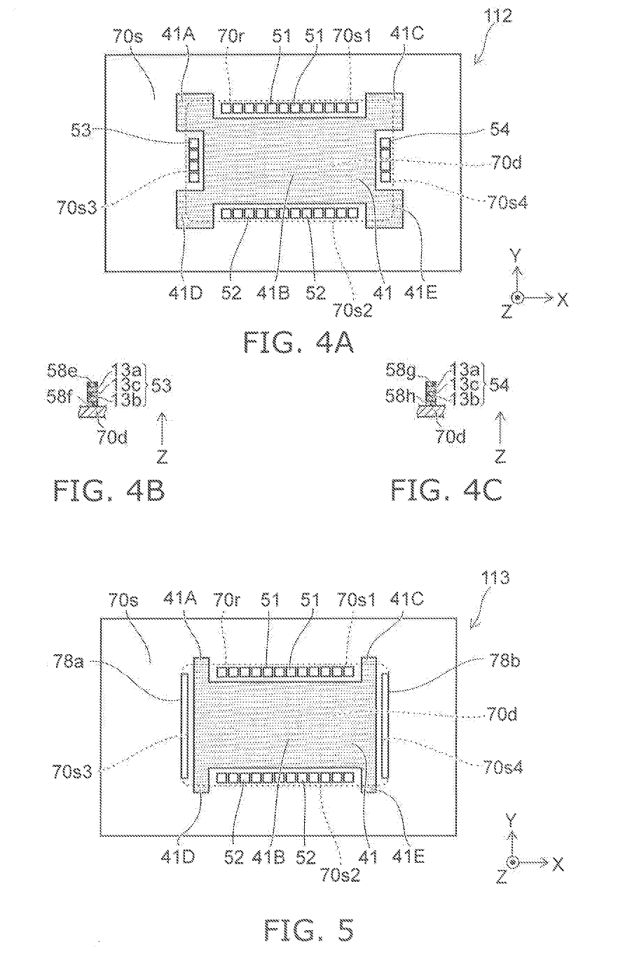

FIG. 4A to FIG. 4C are schematic views illustrating another pressure sensor according to the first embodiment.

FIG. 4A is a plan view corresponding to FIG. 1D. FIG. 4B and FIG. 4C are schematic views illustrating portions of the pressure sensor.

In the other pressure sensor 112 according to the embodiment as shown in FIG. 4A, a third sensing element 53 and a fourth sensing element 54 are provided in addition to the first sensing element 51 and the second sensing element 52. The first structure body 41 further includes a fourth region 41D and a fifth region 41E in addition to the first region 41A, the second region 41B, and the third region 41C.

The third sensing element 53 and the fourth sensing element 54 are fixed to the film portion 70d. In the example, the multiple third sensing elements 53 and the multiple fourth sensing elements 54 are provided.

As shown in FIG. 4B, one of the multiple third sensing elements 53 includes a third magnetic layer 13a, a third opposing magnetic layer 13b, and a third intermediate layer 13c. The third opposing magnetic layer 13b is provided between the third magnetic layer 13a and the film portion 70d. The third intermediate layer 13c is provided between the third magnetic layer 13a and the third opposing magnetic layer 13b.

In the example, the third sensing element 53 further includes a fifth conductive layer 58e and a sixth conductive layer 58f. The third magnetic layer 13a is positioned between the fifth conductive layer 58e and the sixth conductive layer 58f. The third opposing magnetic layer 13b is positioned between the sixth conductive layer 58f and the third magnetic layer 13a.

As shown in FIG. 4C, one of the multiple fourth sensing elements 54 includes a fourth magnetic layer 14a, a fourth opposing magnetic layer 14b, and a fourth intermediate layer 14c. The fourth opposing magnetic layer 14b is provided between the fourth magnetic layer 14a and the film portion 70d. The fourth intermediate layer 14c is provided between the fourth magnetic layer 14a and the fourth opposing magnetic layer 14b.

In the example, the fourth sensing element 54 further includes a seventh conductive layer 58g and an eighth conductive layer 58h. The fourth magnetic layer 14a is positioned between the seventh conductive layer 58g and the eighth conductive layer 58h. The fourth opposing magnetic layer 14b is positioned between the eighth conductive layer 58h and the fourth magnetic layer 14a.

In the example, at least a portion of the second region 41B of the structure body 41 is between the third sensing element 53 and the fourth sensing element 54.

The outer edge 70r of the film portion 70d includes the first side 70s1, the second side 70s2, the third side 70s3, and the fourth side 70s4. The first side 70s1 and the second side 70s2 extend along the second direction (e.g., the X-axis direction). The third direction (e.g., the Y-axis direction) from the second side 70s2 toward the first side 70s1 crosses a plane (e.g., the Z-X plane) including the first direction and the second direction. The third side 70s3 and the fourth side 70s4 extend along the third direction (the Y-axis direction). The direction from the third side 70s3 toward the fourth side 70s4 is aligned with the second direction (the X-axis direction).

The multiple first sensing elements 51 are arranged along the first side 70s1. The multiple second sensing elements 52 are arranged along the second side 70s2. The multiple third sensing elements 53 are arranged along the third side 70s3. The multiple fourth sensing elements 54 are arranged along the fourth side 70s4.

As described above, the second region 41B of the structure body 41 overlaps the film portion 70d in the first direction (the Z-axis direction). The first region 41A, the third region 41C, the fourth region 41D, and the fifth region 41E overlap the support body 70s in the first direction (the Z-axis direction). The first region 41A, the third region 41C, the fourth region 41D, and the fifth region 41E are provided to correspond to the four corner portions of the film portion 70d.

At least a portion of the first sensing element 51 is between the first region 41A and the third region 41C in a direction (e.g., the X-axis direction) crossing the first direction. At least a portion of the second sensing element 52 is between the fourth region 41D and the fifth region 41E in a direction (e.g., the X-axis direction) crossing the first direction. At least a portion of the third sensing element 53 is between the first region 41A and the fourth region 41D in a direction (e.g., the Y-axis direction) crossing the first direction. At least a portion of the fourth sensing element 54 is between the third region 41C and the fifth region 41E in a direction (e.g., the Y-axis direction) crossing the first direction.

For example, the second magnetic layer 12a, the third magnetic layer 13a, and the fourth magnetic layer 14a have configurations (including the materials) similar to that of the first magnetic layer 11a. The second opposing magnetic layer 12b, the third opposing magnetic layer 13b, and the fourth opposing magnetic layer 14b have configurations (including the materials) similar to that of the first opposing magnetic layer 11b. The second intermediate layer 12c, the third intermediate layer 13c, and the fourth intermediate layer 14c have configurations (including the materials) similar to that of the first intermediate layer 11c.

FIG. 5 is a schematic plan view illustrating another pressure sensor according to the first embodiment.

FIG. 5 is a plan view corresponding to FIG. 1D. In the other pressure sensor 113 according to the embodiment as shown in FIG. 5, slits (a first slit 78a and a second slit 78b) are provided in the film portion 70d. For example, a portion of the film portion 70d is separated from the support body 70s. Thus, the slits may be provided.

The portion of the structure body 41 between the first region 41A and the second region 41B overlaps a portion of the first side 70s1. For example, a portion between the third region 41C and the second region 41B overlaps another portion of the first side 70s1. A portion between the fourth region 41D and the second region 41B overlaps a portion of the second side 70s2. For example, a portion between the fifth region 41E and the second region 41B overlaps another portion of the second side 70s2.

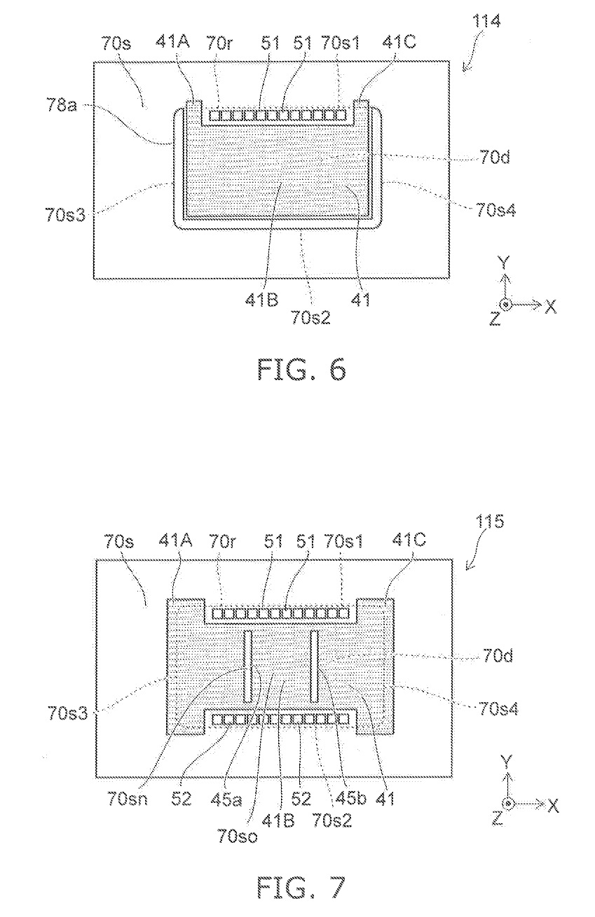

FIG. 6 is a schematic plan view illustrating another pressure sensor according to the first embodiment.

FIG. 6 is a plan view corresponding to FIG. 1D. In the other pressure sensor 114 according to the embodiment as shown in FIG. 6, a slit (the first slit 78a) is provided in the film portion 70d. The slit is provided along three sides of the film portion 70d. The film portion 70d has a “cantilever beam” configuration.

A portion of the structure body 41 between the first region 41A and the second region 41B overlaps a portion of the first side 70s1. For example, a portion between the third region 41C and the second region 41B overlaps another portion of the first side 70s1.

FIG. 7 is a schematic plan view illustrating another pressure sensor according to the first embodiment.

FIG. 7 is a plan view corresponding to FIG. 1D. In the other pressure sensor 115 according to the embodiment as shown in FIG. 7, slits (a first structure body slit 45a, a second structure body slit 45b, etc.) are provided in a portion of the structure body 41.

For example, the film portion 70d includes a portion 70so overlapping the structure body 41 in the first direction (the Z-axis direction), and a portion 70sn not overlapping the structure body 41 in the first direction. The portion of the structure body 41 where the slits are provided corresponds to the non-overlapping portion 70sn. The portion where the slits are not provided corresponds to the overlapping portion 70so.

In the example, the overlapping portion 70so is provided around the non-overlapping portion 70sn. The structure body 41 is one continuous body.

The non-overlapping portion 70so extends along the direction (e.g., the third direction, i.e., the Y-axis direction) from the second sensing element 52 toward the first sensing element 51. For example, the slits of the structure body 41 extend along the Y-axis direction.

Such slits may be provided in the film portion. For example, the film portion 70d deforms more easily due to the pressure to be sensed (the sound, etc.). For example, the sensitivity can be adjusted.

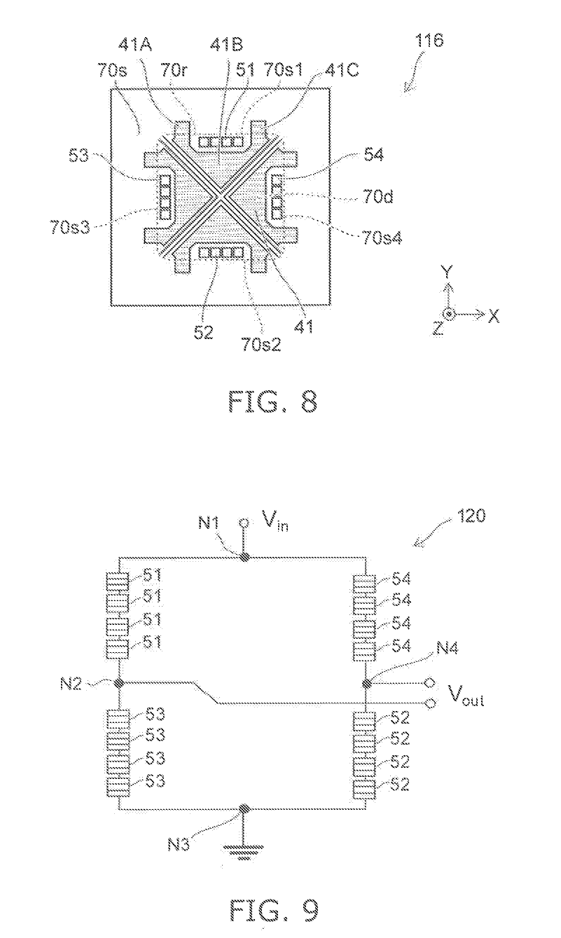

FIG. 8 is a schematic plan view illustrating another pressure sensor according to the first embodiment.

FIG. 8 is a plan view corresponding to FIG. 1D. As shown in FIG. 8, multiple film portions 70d are provided in the other pressure sensor 116 according to the embodiment. Four film portions 70d are provided in the example. Each of the multiple film portions 70d has a “cantilever beam” configuration.

Multiple structure bodies 41 are provided in the example. The multiple structure bodies 41 are provided to correspond respectively to the multiple film portions 70d.

One (each) of the multiple structure bodies 41 includes the first region 41A recited above and the second region 41B recited above. In the example, one (each) of the multiple structure bodies 41 further includes the third region 41C recited above.

FIG. 9 is a schematic view illustrating a portion of another pressure sensor according to the first embodiment.

In the pressure sensor 120 according to the embodiment as shown in FIG. 9, the processor 68 may include a bridge circuit (e.g., a Wheatstone bridge circuit).

For example, the multiple first sensing elements 51, the multiple second sensing elements 52, the multiple third sensing elements 53, and the multiple fourth sensing elements 54 are provided as in the pressure sensor 112.

As shown in FIG. 9, the multiple first sensing elements 51 are connected in series to each other. The multiple second sensing elements 52 are connected in series to each other. The multiple third sensing elements 53 are connected in series to each other. The multiple fourth sensing elements 54 are connected in series to each other.

One end of the multiple first sensing elements 51 connected in series is electrically connected to a first node N1 of the bridge circuit. The other end of the multiple first sensing elements 51 connected in series is electrically connected to a second node N2 of the bridge circuit. One end of the multiple third sensing elements 53 connected in series is electrically connected to the second node N2. The other end of the multiple third sensing elements 53 connected in series is electrically connected to a third node N3 of the bridge circuit. One end of the multiple second sensing elements 52 connected in series is electrically connected to the third node N3. The other end of the multiple second sensing elements 52 connected in series is electrically connected to a fourth node N4 of the bridge circuit. One end of the multiple fourth sensing elements 54 connected in series is electrically connected to the fourth node N4. The other end of the multiple fourth sensing elements 54 connected in series is electrically connected to the first node N1.

The noise is suppressed by connecting the sensing elements using such a bridge circuit. It is possible to sense with high sensitivity.

Examples of the sensing elements used in the first embodiment will now be described. In the following description, the notation “material A/material B” indicates a state in which a layer of material B is provided on a layer of material A.

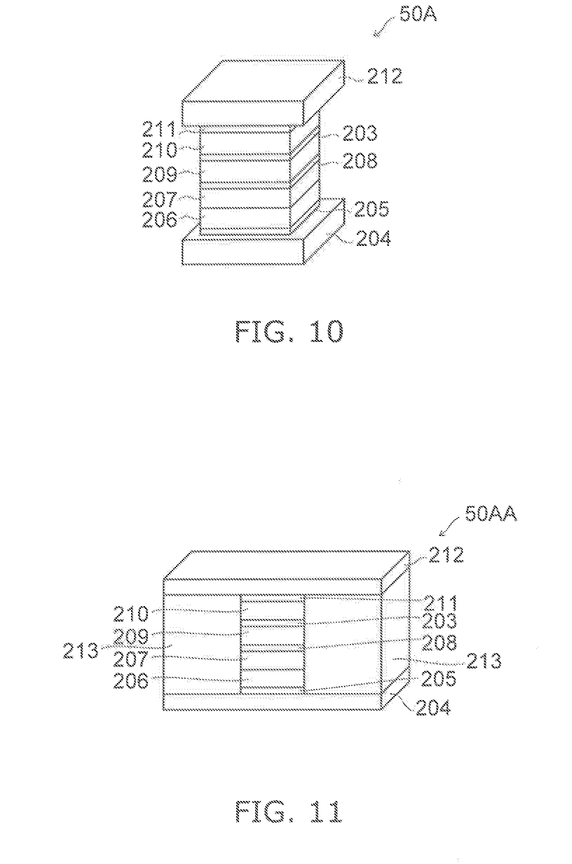

FIG. 10 is a schematic perspective view illustrating a portion of the pressure sensor according to the embodiment.

In the sensing element 50A as shown in FIG. 10, a lower electrode 204, a foundation layer 205, a pinning layer 206, a second fixed magnetic layer 207, a magnetic coupling layer 208, a first fixed magnetic layer 209, an intermediate layer 203, a free magnetic layer 210, a capping layer 211, and an upper electrode 212 are arranged in this order. The sensing element 50A is, for example, a bottom spin-valve type.

The foundation layer 205 includes, for example, a stacked film of tantalum and ruthenium (Ta/Ru). The thickness (the length in the Z-axis direction) of the Ta layer is, for example, 3 nanometers (nm). The thickness of the Ru layer is, for example, 2 nm. The pinning layer 206 includes, for example, an IrMn-layer having a thickness of 7 nm. The second fixed magnetic layer 207 includes, for example, a Co₇₅Fe₂₅ layer having a thickness of 2.5 nm. The magnetic coupling layer 208 includes, for example, a Ru layer having a thickness of 0.9 nm. The first fixed magnetic layer 209 includes, for example, a Co₄₀Fe₄₀B₂₀ layer having a thickness of 3 nm. The intermediate layer 203 includes, for example, a MgO layer having a thickness of 1.6 nm. The free magnetic layer 210 includes, for example, Co₄₀Fe₄₀B₂₀ having a thickness of 4 nm. The capping layer 211 includes, for example, Ta/Ru. The thickness of the Ta layer is, for example, 1 nm. The thickness of the Ru layer is, for example, 5 nm.

The lower electrode 204 and the upper electrode 212 include, for example, at least one of aluminum (Al), an aluminum copper alloy (Al—Cu), copper (Cu), silver (Ag), gold (Au), a copper-silver alloy (Cu—Ag), platinum (Pt), or palladium (Pd). By using such a material having a relatively small electrical resistance as the lower electrode 204 and the upper electrode 212, the current can be caused to flow efficiently through the sensing element 50A. The lower electrode 204 and the upper electrode 212 include nonmagnetic materials. The lower electrode 204 and the upper electrode 212 may include the at least one of the elements recited above and another element (an added element). The added element is, for example, Si. The lower electrode 204 and the upper electrode 212 may include, for example, a Corson alloy (Cu—Ni—Si), etc.

The lower electrode 204 and the upper electrode 212 may include, for example, a foundation layer (not illustrated) for the lower electrode 204 and the upper electrode 212, a capping layer (not illustrated) for the lower electrode 204 and the upper electrode 212, and a layer of at least one of Al, Al—Cu, Cu, Ag, Au, Cu—Ag, Pt, or Pd provided between the foundation layer and the capping layer. For example, the lower electrode 204 and the upper electrode 212 include tantalum (Ta)/copper (Cu)/tantalum (Ta), etc. For example, by using Ta as the foundation layer of the lower electrode 204 and the upper electrode 212, the adhesion between the substrate (e.g., the film portion 70d) and the lower electrode 204 and between the substrate and the upper electrode 212 improves. Titanium (Ti), titanium nitride (TiN), etc., may be used as the foundation layer for the lower electrode 204 and the upper electrode 212. The lower electrode 204 and the upper electrode 212 may include a foundation layer, a capping layer, and a layer of a Corson alloy provided between the foundation layer and the capping layer. The capping layer recited above may include, for example, at least one selected from the group consisting of tantalum nitride (TaN), a tantalum-molybdenum alloy (Ta—Mo), tungsten, and a tungsten-molybdenum alloy (W—Mo).

By using Ta as the capping layer of the lower electrode 204 and the upper electrode 212, the oxidization of the copper (Cu), etc., under the capping layer is suppressed. Titanium (Ti), titanium nitride (TiN), etc., may be used as the capping layer for the lower electrode 204 and the upper electrode 212.

The foundation layer 205 includes, for example, a stacked structure including a buffer layer (not illustrated) and a seed layer (not illustrated). For example, the buffer layer relaxes the roughness of the surfaces of the lower electrode 204, the film portion 70d, etc., and improves the crystallinity of the layers stacked on the buffer layer. For example, at least one selected from the group consisting of tantalum (Ta), titanium (Ti), vanadium (V), tungsten (W), zirconium (Zr) hafnium (Hf), and chrome (Cr) is used as the buffer layer. An alloy that includes at least one material selected from these materials may be used as the buffer layer.

It is favorable for the thickness of the buffer layer of the foundation layer 205 to be not less than 1 nm and not more than 10 nm. It is more favorable for the thickness of the buffer layer to be not less than 1 nm and not more than 5 nm. In the case where the thickness of the buffer layer is too thin, the buffering effect is lost. In the case where the thickness of the buffer layer is too thick, the thickness of the sensing element 50A becomes excessively thick. The seed layer is formed on the buffer layer; and, for example, the seed layer has a buffering effect. In such a case, the buffer layer may be omitted. The buffer layer includes for example, a Ta layer having a thickness of 3 nm.

The seed layer of the foundation layer 205 controls the crystal orientation of the layers stacked on the seed layer. The seed layer controls the crystal grain size of the layers stacked on the seed layer. As the seed layer, a metal having a fcc structure (face-centered cubic structure), a hcp structure (hexagonal close-packed structure), a bcc structure (body-centered cubic structure), or the like is used.

For example, the crystal orientation of the spin-valve film on the seed layer can be set to the fcc (111) orientation by using, as the seed layer of the foundation layer 205, ruthenium (Ru) having a hcp structure, NiFe having a fcc structure, or Cu having a fcc structure. The seed layer includes, for example, a Cu layer having a thickness of 2 nm or a Ru layer having a thickness of 2 nm. To increase the crystal orientation of the layers formed on the seed layer, it is favorable for the thickness of the seed layer to be not less than 1 nm and not more than 5 nm. It is more favorable for the thickness of the seed layer to be not less than 1 nm and not more than 3 nm. Thereby, the function as a seed layer that improves the crystal orientation is realized sufficiently.

On the other hand, for example, the seed layer may be omitted in the case where it is unnecessary for the layers formed on the seed layer to have a crystal orientation (e.g., in the case where an amorphous free magnetic layer is formed, etc.). For example, a Ru layer having a thickness of 2 nm is used as the seed layer.

For example, the pinning layer 206 provides unidirectional anisotropy to the second fixed magnetic layer 207 (the ferromagnetic layer) formed on the pinning layer 206 and fixes the magnetization of the second fixed magnetic layer 207. The pinning layer 206 includes, for example, an antiferromagnetic layer. The pinning layer 206 includes, for example, at least one selected from the group consisting of Ir—Mn, Pt—Mn, Pd—Pt—Mn, Ru—Mn, Rh—Mn, Ru—Rh—Mn, Fe—Mn, Ni—Mn, Cr—Mn—Pt, and Ni—O. An alloy may be used in which an added element is further added to the at least one selected from the group consisting of Ir—Mn, Pt—Mn, Pd—Pt—Mn, Ru—Mn, Rh—Mn, Ru—Rh—Mn, Fe—Mn, Ni—Mn, Cr—Mn—Pt, and Ni—O. The thickness of the pinning layer 206 is set appropriately. Thereby, for example, unidirectional anisotropy of sufficient strength is provided.

For example, heat treatment is performed while applying a magnetic field. Thereby, for example, the magnetization of the ferromagnetic layer contacting the pinning layer 206 is fixed. The magnetization of the ferromagnetic layer contacting the pinning layer 206 is fixed in the direction of the magnetic field applied in the heat treatment. For example, the heat treatment temperature (the annealing temperature) is not less than the magnetization pinning temperature of the antiferromagnetic material included in the pinning layer 206. In the case where an antiferromagnetic layer including Mn is used, there are cases where the MR ratio decreases due to the Mn diffusing into layers other than the pinning layer 206. It is desirable for the heat treatment temperature to be set to be not more than the temperature at which the diffusion of Mn occurs. The heat treatment temperature is, for example, not less than 200° C. and not more than 500° C. Favorably, the heat treatment temperature is, for example, not less than 250° C. and not more than 400° C.

In the case where PtMn or PdPtMn is used as the pinning layer 206, it is favorable for the thickness of the pinning layer 206 to be not less than 8 nm and not more than 20 nm. It is more favorable for the thickness of the pinning layer 206 to be not less than 10 nm and not more than 15 nm. In the case where IrMn is used as the pinning layer 206, unidirectional anisotropy can be provided using a thickness that is thinner than the case where PtMn is used as the pinning layer 206. In such a case, it is favorable for the thickness of the pinning layer 206 to be not less than 4 nm and not more than 18 nm. It is more favorable for the thickness of the pinning layer 206 to be not less than 5 nm and not more than 15 nm. The pinning layer 206 includes, for example, an Ir₂₂Mn₇₈ layer having a thickness of 7 nm.

A hard magnetic layer may be used as the pinning layer 206. For example, Co—Pt, Fe—Pt, Co—Pd, Fe—Pd, etc., may be used as the hard magnetic layer. For example, the magnetic anisotropy and the coercivity are relatively high for these materials. These materials are hard magnetic materials. An alloy in which an added element is further added to Co—Pt, Fe—Pt, Co—Pd, or Fe—Pd may be used as the pinning layer 206. For example, CoPt (the proportion of Co being not less than 50 at. % and not more than 85 at. %), (CoₓPt₁₀₀₋ₓ)_{100-y}Cr_{y} (x being not less than 50 at. % and not more than 85 at. %, and y being not less than 0 at % and not more than 40 at. %), FePt (the proportion of Pt being not less than 40 at. % and not more than 60 at. %), etc., may be used.

The second fixed magnetic layer 207 includes, for example, a CoₓFe₁₀₀₋ₓ alloy (x being not less than 0 at. % and not more than 100 at. %) or a NiₓFe₁₀₀₋ₓ alloy (x being not less than 0 at. % and not more than 100 at. %). These materials may include a material to which a nonmagnetic element is added. For example, at least one selected from the group consisting of Co, Fe, and Ni is used as the second fixed magnetic layer 207. An alloy that includes at least one material selected from these materials may be used as the second fixed magnetic layer 207. Also, a (CoₓFe_{100 -x})_{100-y}B_{y} alloy (x being not less than 0 at. % and not more than 100 at. %, and y being not less than 0 at. % and not more than 30 at. %) may be used as the second fixed magnetic layer 207. By using an amorphous alloy of (CoₓFe₁₀₀₋ₓ)_{100-y}B_{y} as the second fixed magnetic layer 207, the fluctuation of the characteristics of the sensing element 50A can be suppressed even in the case where the sizes of the sensing elements are small.

For example, it is favorable for the thickness of the second fixed magnetic layer 207 to be not less than 1.5 nm and not more than 5 nm. Thereby, for example, the strength of the unidirectional anisotropic magnetic field due to the pinning layer 206 can be stronger. For example, the strength of the antiferromagnetic coupling magnetic field between the second fixed magnetic layer 207 and the first fixed magnetic layer 209 via the magnetic coupling layer formed on the second fixed magnetic layer 207 can be stronger. For example, it is favorable for the magnetic thickness (the product (Bs·t) of a saturation magnetization Bs and a thickness t) of the second fixed magnetic layer 207 to be substantially equal to the magnetic thickness of the first fixed magnetic layer 209.

The saturation magnetization of the thin film of Co₄₀Fe₄₀B₂₀ is about 1.9 T (teslas). For example, in the case where a Co₄₀Fe₄₀B₂₀ layer having a thickness of 3 nm is used as the first fixed magnetic layer 209, the magnetic thickness of the first fixed magnetic layer 209 is 1.9 T×3 nm, i.e., 5.7 Tnm. On the other hand, the saturation magnetization of Co₇₅Fe₂₅ is about 2.1 T. The thickness of the second fixed magnetic layer 207 to obtain a magnetic thickness equal to that recited above is 5.7 Tnm/2.1 T, i.e., 2.7 nm. In such a case, it is favorable for a Co₇₅Fe₂₅ layer having a thickness of about 2.7 nm to be included in the second fixed magnetic layer 207. For example, a Co₇₅Fe₂₅ layer having a thickness of 2.5 nm is used as the second fixed magnetic layer 207.

In the sensing element 50A, a synthetic pinned structure that is made of the second fixed magnetic layer 207, the magnetic coupling layer 208, and the first fixed magnetic layer 209 is used. A single pinned structure that is made of one fixed magnetic layer may be used instead. In the case where the single pinned structure is used, for example, a Co₄₀Fe₄₀B₂₀ layer having a thickness of 3 nm is used as the fixed magnetic layer. The same material as the second fixed magnetic layer 207 described above may be used as the ferromagnetic layer included in the fixed magnetic layer having the single pinned structure.

The magnetic coupling layer 208 causes antiferromagnetic coupling to occur between the second fixed magnetic layer 207 and the first fixed magnetic layer 209. The magnetic coupling layer 208 has a synthetic pinned structure. For example, Ru is used as the material of the magnetic coupling layer 208. For example, it is favorable for the thickness of the magnetic coupling layer 208 to be not less than 0.8 nm and not more than 1 nm. A material other than Ru may be used as the magnetic coupling layer 208 if the material causes sufficient antiferromagnetic coupling to occur between the second fixed magnetic layer 207 and the first fixed magnetic layer 209. For example, the thickness of the magnetic coupling layer 208 is set to a thickness not less than 0.8 nm and not more than 1 nm corresponding to the second peak (2nd peak) of RKKY (Ruderman-Kittel-Kasuya-Yosida) coupling. Further, the thickness of the magnetic coupling layer 208 may be set to a thickness not less than 0.3 nm and not more than 0.6 nm corresponding to the first peak (1st peak) of RKKY coupling. For example, Ru having a thickness of 0.9 nm is used as the material of the magnetic coupling layer 208. Thereby, highly reliable coupling is obtained more stably.

The magnetic layer that is included in the first fixed magnetic layer 209 contributes directly to the MR effect. For example, a Co—Fe—B alloy is used as the first fixed magnetic layer 209. Specifically, a (CoₓFe₁₀₀₋ₓ)_{100-y}B_{y} alloy (x being not less than 0 at. % and not more than 100 at. %, and y being not less than 0 at. % and not more than 30 at. %) also may be used as the first fixed magnetic layer 209. For example, the fluctuation between the elements caused by crystal grains can be suppressed even in the case where the size of the sensing element 50A is small by using a (CoₓFe₁₀₀₋ₓ)_{100-y}B_{y} amorphous alloy as the first fixed magnetic layer 209.

The layer (e.g. a tunneling insulating layer (not illustrated)) that is formed on the first fixed magnetic layer 209 can be planarized. The defect density of the tunneling insulating layer can be reduced by the planarization of the tunneling insulating layer. Thereby, a higher MR ratio is obtained with a lower resistance per area. For example, in the case where MgO is used as the material of the tunneling insulating layer, the (100) orientation of the MgO layer that is formed on the tunneling insulating layer can be strengthened by using a (CoₓFe₁₀₀₋ₓ)_{100-y}B_{y} amorphous alloy as the first fixed magnetic layer 209. A higher MR ratio is obtained by increasing the (100) orientation of the MgO layer. The (CoₓFe_{10 -x})_{100-y}B_{y} alloy crystallizes using the (100) plane of the MgO layer as a template when annealing. Therefore, good crystal conformation between the MgO and the (CoₓFe₁₀₀₋ₓ)_{100-y}B_{y} alloy is obtained. A higher MR ratio is obtained by obtaining good crystal conformation.

Other than the Co—Fe—B alloy, for example, an Fe—Co alloy may be used as the first fixed magnetic layer 209.

A higher MR ratio is obtained as the thickness of the first fixed magnetic layer 209 increases. For example, a larger fixed magnetic field is obtained as the thickness of the first fixed magnetic layer 209 decreases. A trade-off relationship between the MR ratio and the fixed magnetic field exists for the thickness of the first fixed magnetic layer 209. In the case where the Co—Fe—B alloy is used as the first fixed magnetic layer 209, it is favorable for the thickness of the first fixed magnetic layer 209 to be not less than 1.5 nm and not more than 5 nm. It is more favorable for the thickness of the first fixed magnetic layer 209 to be not less than 2.0 nm and not more than 4 nm.

Other than the materials described above, the first fixed magnetic layer 209 may include a Co₉₀Fe₁₀ alloy having a fcc structure, Co having a hcp structure, or a Co alloy having a hcp structure. For example, at least one selected from the group consisting of Co, Fe, and Ni is used as the first fixed magnetic layer 209. An alloy that includes at least one material selected from these materials is used as the first fixed magnetic layer 209. For example, a higher MR ratio is obtained by using an FeCo alloy material having a bcc structure, a Co alloy having a cobalt composition of 50% or more, or a material (a Ni alloy) having a Ni composition of 50% or more as the first fixed magnetic layer 209.

For example, a Heusler magnetic alloy layer such as Co₂MnGe, Co₂FeGe, Co₂MnSi, Ce₂FeSi, Co₂MnAl, Co₂FeAl, Co₂MnGa_{0.5}Ge_{0.5}, Co₂FeGa_{0.5}Ge_{0.5}, etc., also may be used as the first fixed magnetic layer 209. For example, a Co₄₀Fe₄₀B₂₀ layer having a thickness of, for example, 3 nm is used as the first fixed magnetic layer 209.

For example, the intermediate layer 203 breaks the magnetic coupling between the first fixed magnetic layer 209 and the free magnetic layer 210.

For example, the material of the intermediate layer 203 includes a metal, an insulator, or a semiconductor. For example, Cu, Au, Ag, or the like is used as the metal. In the case where a metal is used as the intermediate layer 203, the thickness of the intermediate layer is, for example, not less than about 1 nm and not more than about 7 nm. For example, magnesium oxide (MgO, etc.), aluminum oxide (Al₂O₃, etc.), titanium oxide (TiO, etc.), zinc oxide (ZnO, etc.), gallium oxide (Ga—O), or the like is used as the insulator or the semiconductor. In the case where the insulator or the semiconductor is used as the intermediate layer 203, the thickness of the intermediate layer 203 is, for example, not less than about 0.6 nm and not more than about 2.5 nm. For example, a CCP (Current-Confined-Path) spacer layer may be used as the intermediate layer 203. In the case where a CCP spacer layer is used as the spacer layer, for example, a structure is used in which a copper (Cu) metal path is formed inside an insulating layer of aluminum oxide (Al₂O₃). For example, a MgO layer having a thickness of 1.6 nm is used as the intermediate layer.

The free magnetic layer 210 includes a ferromagnet material. For example, the free magnetic layer 210 includes a ferromagnet material including Fe, Co, and Ni. For example, an FeCo alloy, a NiFe alloy, or the like is used as the material of the free magnetic layer 210. Further, the free magnetic layer 210 includes a Co—Fe—B alloy, an Fe—Co—Si—B alloy, an Fe—Ga alloy having a large λs (magnetostriction constant), an Fe—Co—Ga alloy, a Tb—M—Fe alloy, a Tb—M1—Fe—M2 alloy, an Fe—M3—M4—B alloy, Ni, Fe—Al, ferrite, etc. For example, the λs (the magnetostriction constant) is large for these materials. In the Tb—M—Fe alloy recited above, M is at least one selected from the group consisting of Sm, Eu, Gd, Dy, Ho, and Er. In the Tb—M1—Fe—M2 alloy recited above, M1 is at least one selected from the group consisting of Sm, Eu, Gd, Dy, Ho, and Er. M2 is at least one selected from the group consisting of Ti, Cr, Mn, Co, Cu, Nb, Mo, W, and Ta. In the Fe—M3—M4—B alloy recited above, M3 is at least one selected from the group consisting of Ti, Cr, Mn, Co, Cu, Nb, Mo, W, and Ta, M4 is at least one selected from the group consisting of Ce, Pr, Nd, Sm, Tb, Dy and Er. Fe₃O₄, (FeCo)₃O₄, etc., are examples of the ferrite recited above. The thickness of the free magnetic layer 210 is, for example, 2 nm or more.

The free magnetic layer 210 may include a magnetic material including boron. The free magnetic layer 210 may include, for example, an alloy including boron (B) and at least one element selected from the group consisting of Fe, Co, and Ni. The free magnetic layer 210 includes, for example, a Co—Fe—B alloy or an Fe—B alloy. For example, a Co₄₀Fe₄₀B₂₀ alloy is used. Ga, Al, Si, W, etc., may be added in the case where the free magnetic layer 210 includes an alloy including boron (B) and at least one element selected from the group consisting of Fe, Co, and Ni. For example, high magnetostriction is promoted by adding these elements. For example, an Fe—Ga—B alloy, an Fe—Co—Ga—B alloy, or an Fe—Co—Si—B alloy may be used as the free magnetic layer 210. By using such a magnetic material containing boron, the coercivity (Hc) of the free magnetic layer 210 is low; and the change of the magnetization direction for the strain is easy. Thereby, high sensitivity is obtained.

It is favorable for the boron concentration (e.g., the composition ratio of boron) of the free magnetic layer 210 to be 5 at. % (atomic percent) or more. Thereby, an amorphous structure is easier to obtain. It is favorable for the boron concentration of the free magnetic layer to be 35 at. % or less. For exempla, the magnetostriction constant decreases when the boron concentration is too high. For example, it is favorable for the boron concentration of the free magnetic layer to be not less than 5 at. % and not more than 35 at. %; and it is more favorable to be not less than 10 at. % and not more than 30 at. %.

In the case where a portion of the magnetic layer of the free magnetic layer 210 includes Fe_{1-y}B_{y} (0<y≤0.3) or (Fe_{z}X_{1-z})_{1-y}B_{y} (X being Co or Ni, 0.8≤z<1, and 0<y≤0.3), it is easy to realize both a large magnetostriction constant λ and a low coercivity. Therefore, this is particularly favorable from the perspective of obtaining a high gauge factor. For example, Fe₈₀B₂₀ (4 nm) is used as the free magnetic layer 210. Co₄₀Fe₄₀B₂₀ (0.5 nm)/Fe₈₀B₂₀ (4 nm) is used as the free magnetic layer.

The free magnetic layer 210 may have a multilayered structure. In the case where a tunneling insulating layer of MgO is used as the intermediate layer 203, it is favorable to provide a layer of a Co—Fe—B alloy at the portion of the free magnetic layer 210 contacting the intermediate layer 203. Thereby, a high magnetoresistance effect is obtained. In such a case, a layer of a Co—Fe—B alloy is provided on the intermediate layer 203; and another magnetic material that has a large magnetostriction constant is provided on the layer of the Co—Fe—B alloy, In the case where the free magnetic layer 210 has the multilayered structure, for example, the free magnetic layer 210 includes Co—Fe—B (2 nm)/Fe—Co—Si—B (4 nm), etc.

The capping layer 211 protects the layers provided under the capping layer 211. The capping layer 211 includes, for example, multiple metal layers. The capping layer 211 includes, for example, a two-layer structure (Ta/Ru) of a Ta layer and a Ru layer. The thickness of the Ta layer is, for example, 1 nm; and the thickness of the Ru layer is, for example, 5 nm. As the capping layer 211, another metal layer may be provided instead of the Ta layer and/or the Ru layer. The configuration of the capping layer 211 is arbitrary. For example, a nonmagnetic material is used as the capping layer 211. Another material may be used as the capping layer 211 as long as the material can protect the layers provided under the capping layer 211.

In the case where the free magnetic layer 210 includes a magnetic material containing boron, a diffusion suppression layer (not illustrated) of an oxide material and/or a nitride material may be provided between the free magnetic layer 210 and the capping layer 211. Thereby, for example, the diffusion of boron is suppressed. By using the diffusion suppression layer including an oxide layer or a nitride layer, the diffusion of the boron included in the free magnetic layer 210 can be suppressed; and the amorphous structure of the free magnetic layer 210 can be maintained. As the oxide material and/or the nitride material included in the diffusion suppression layer, for example, an oxide material or a nitride material including an element such as Mg, Al, Si, Ti, V, Cr, Mn, Fe, Co, Ni, Cu, Zn, Zr, Nb, Mo, Ru, Rh, Pd, Ag, Hf, Ta, Sn, Cd, Ga, or the like is used. The diffusion suppression layer is a layer that does not contribute to the magnetoresistance effect. It is favorable for the resistance per area of the diffusion suppression layer to be low. For example, it is favorable for the resistance per area of the diffusion suppression layer to be set to be lower than the resistance per area of the intermediate layer that contributes to the magnetoresistance effect. From the perspective of reducing the resistance per area of the diffusion suppression layer, it is favorable for the diffusion suppression layer to be an oxide or a nitride of Mg, Ti, V, Zn, Sn, Cd, and Ga. The barrier height is low for these materials. It is favorable to use an oxide having a stronger chemical bond to suppress the diffusion of boron. For example, a MgO layer of 1.5 nm is used. Oxynitrides are included in one of the oxide or the nitride.

In the case where the diffusion suppression layer includes an oxide or a nitride, it is favorable for the thickness of the diffusion suppression layer to be, for example, 0.5 nm or more. Thereby, the diffusion suppression function of the boron is realized sufficiently. It is favorable for the thickness of the diffusion suppression layer to be 5 nm or less. Thereby, for example, a low resistance per area is obtained. It is favorable for the thickness of the diffusion suppression layer to be not less than 0.5 nm and not more than 5 nm; and it is favorable to be not less than 1 nm and not more than 3 nm.

At least one selected from the group consisting of magnesium (Mg), silicon (Si), and aluminum (Al) may be used as the diffusion suppression layer. A material that includes these light elements is used as the diffusion suppression layer. These light elements produce compounds by bonding with boron. For example, at least one of a Mg—B compound, an Al—B compound, or a Si—B compound is formed at the portion including the interface between the diffusion suppression layer and the free magnetic layer 210. These compounds suppress the diffusion of boron.

Another metal layer, etc., may be inserted between the diffusion suppression layer and the free magnetic layer 210. In the case where the distance between the diffusion suppression layer and the free magnetic layer 210 is too long, boron diffuses between the diffusion suppression layer and the free magnetic layer 210; and the boron concentration in the free magnetic layer 210 undesirably decreases. Therefore, it is favorable for the distance between the diffusion suppression layer and the free magnetic layer 210 to be 10 nm or less; and it is more favorable to be 3 nm or less.

FIG. 11 is a schematic perspective view illustrating a portion of another pressure sensor according to the embodiment.

As shown in FIG. 11, other than an insulating layer 213 being provided, the sensing element SOAR is similar to the sensing element 50A. The insulating layer 213 is provided between the lower electrode 204 and the upper electrode 212. The insulating layer 213 is arranged with the free magnetic layer 210 and the first fixed magnetic layer 209 in a direction crossing the direction connecting the lower electrode 204 and the upper electrode 212. Portions other than the insulating layer 213 are similar to those of the sensing element 50A; and a description is therefore omitted.

The insulating layer 213 includes, for example, at least one selected from the group consisting of aluminum oxide (e.g., Al₂O₃), silicon oxide (e.g., SiO₂), and silicon nitride (e.g., Si₃N4), etc. The leakage current of the sensing element 50AA is suppressed by the insulating layer 213. The insulating layer 213 may be provided in the sensing elements described below.

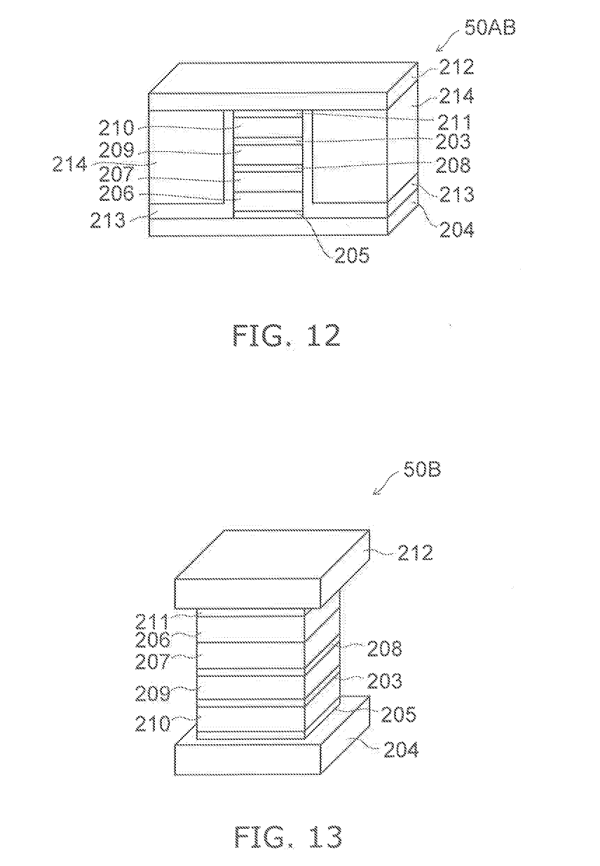

FIG. 12 is a schematic perspective view illustrating a portion of another pressure sensor according to the embodiment.

As shown in FIG. 12, a hard bias layer 214 is further provided in the sensing element 50AB. Otherwise, the sensing element 50AB is similar to the sensing element 50A. The hard bias layer 214 is provided between the lower electrode 204 and the upper electrode 212. The free magnetic layer 210 and the first fixed magnetic layer 209 are disposed between two portions of the hard bias layer 214 in a direction crossing the direction connecting between the lower electrode 204 and the upper electrode 212. Otherwise, the sensing element 50AB is similar to the sensing element 50AA.

The hard bias layer 214 sets the magnetization direction of the free magnetic layer 210 by the magnetization of the hard bias layer 214. The magnetization direction of the free magnetic layer 210 is set to the desired direction by the hard bias layer 214 in a state in which pressure from the outside is not applied to the film portion 70d.

The hard bias layer 214 includes, for example, Co—Pt, Fe—Pt, Co—Pd, Fe Pd, etc. For example, the magnetic anisotropy and the coercivity are relatively high for these materials. These materials are, for example, hard magnetic materials. The hard bias layer 214 may include, for example, an alloy in which an added element is further added to Co—Pt, Fe—Pt, Co—Pd, or Fe—Pd. The hard bias layer 214 may include, for example, CoPt (the proportion of Co being not less than 50 at. % and not more than 85 at. %), (CoₓPt₁₀₀₋ₓ)_{100-y}Cr_{y} (x being not less than 50 at. % and not more than 85 at. %, and y being not less than 0 at. % and not more than 40 at. %), FePt (the proportion of Pt being not less than 40 at. % and not more than 60 at. %), etc. In the case where such a material is used, by applying an external magnetic field that is larger than the coercivity of the hard bias layer 214, the direction of the magnetization of the hard bias layer 214 is set (fixed) in the direction in which the external magnetic field is applied. The thickness (e.g., the length along the direction from the lower electrode 204 toward the upper electrode) of the hard bias layer 214 is, for example, not less than 5 nm and not more than 50 nm.

In the case where the insulating layer 213 is disposed between the lower electrode 204 and the upper electrode 212, at least one selected from the group consisting of SiOₓ, AlOₓ, and SiNₓ is used as the material of the insulating layer 213. A not-illustrated foundation layer may be further provided between the insulating layer 213 and the hard bias layer 214. Cr, Fe—Co, or the like is used as the material of the foundation layer for the hard bias layer 214 in the case where the hard bias layer 214 includes a hard magnetic material such as Co—Pt, Fe—Pt, Co—Pd, Fe—Pd, etc.

The hard bias layer 214 may have a structure of being stacked with a not-illustrated pinning layer for the hard bias layer. In such a case, the direction of the magnetization of the hard bias layer 214 can be set (fixed) by the exchange coupling of the hard bias layer 214 and the pinning layer for the hard bias layer. In such a case, the hard bias layer 214 includes a ferromagnetic material of at least one of Fe, Co, or Ni or an alloy including at least one type of these elements. In such a case, the hard bias layer 214 includes, for example, a CoₓFe₁₀₀₋ₓ alloy (x being not less than 0 at. % and not more than 100 at. %), a NiₓFe₁₀₀₋ₓ alloy (x being not less than 0 at. % and not more than 100 at. %), or a material in which a nonmagnetic element is added to these alloys. A material that is similar to the first fixed magnetic layer 209 recited above is used as the hard bias layer 214. The pinning layer for the hard bias layer includes a material similar to the pinning layer 206 inside the sensing element 50A recited above. In the case where the pinning layer for the hard bias layer is provided, a foundation layer similar to the material included in the foundation layer 205 may be provided under the pinning layer for the hard bias layer. The pinning layer for the hard bias layer may be provided at a lower portion or at an upper portion of the hard bias layer. In such a case, the magnetization direction of the hard bias layer 214 is determined by heat treatment in a magnetic field similarly to the pinning layer 206.

The hard bias layer 214 and the insulating layer 213 recited above are applicable to any sensing element according to the embodiment. By using the stacked structure of the hard bias layer 214 and the pinning layer for the hard bias layer, the orientation of the magnetization of the hard bias layer 214 can be maintained easily even when a large external magnetic field is applied to the hard bias layer 214 for a short period of time.

FIG. 13 is a schematic perspective view illustrating a portion of another pressure sensor according to the embodiment.

In the sensing element 50B as shown in FIG. 13, the lower electrode 204, the foundation layer 205, the free magnetic layer 210, the intermediate layer 203, the first fixed magnetic layer 209, the magnetic coupling layer 208, the second fixed magnetic layer 207, the pinning layer 206, the capping layer 211, and the upper electrode 212 are stacked in order. The sensing element 50B is, for example, a top spin-valve type.

The foundation layer 205 includes, for example, a stacked film of tantalum and copper (Ta/Cu). The thickness (the length in the Z-axis direction) of the Ta layer is, for example, 3 nm. The thickness of the Cu layer is, for example, 5 nm. The free magnetic layer 210 includes, for example, Co₄₀Fe₄₀B₂₀ having a thickness of 4 nm. The intermediate layer 203 includes, for example, a MgO layer having a thickness of 1.6 nm. The first fixed magnetic layer 209 includes, for example, Co₄₀Fe₄₀B₂₀/Fe₅₀Co₅₀. The thickness of the Co₄₀Fe₄₀B₂₀ layer is, for example, 2 nm. The thickness of the Fe₅₀Co₅₀ layer is, for example, 1 nm. The magnetic coupling layer 208 includes, for example, a Ru layer having a thickness of 0.9 nm. The second fixed magnetic layer 207 includes, for example, a Co₇₅Fe₂₅ layer having a thickness of 2.5 nm. The pinning layer 206 includes, for example, an IrMn-layer having a thickness of 7 nm. The capping layer 211 includes, for example, Ta/Ru. The thickness of the Ta layer is, for example, 1 nm. The thickness of the Ru layer is, for example, 5 nm.

The materials of the layers included in the sensing element 50B may be the vertically inverted materials of the layers included in the sensing element 50A. The diffusion suppression layer recited above may be provided between the foundation layer 205 and the free magnetic layer 210 of the sensing element 50B.

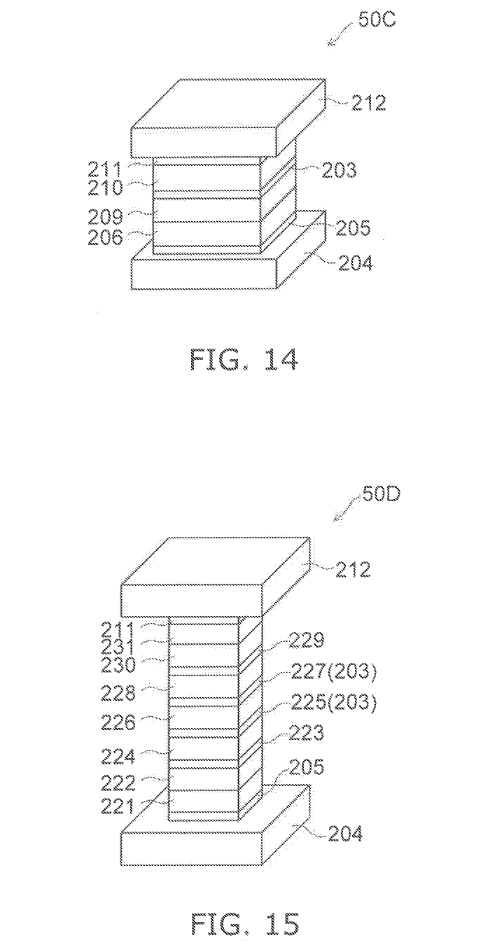

FIG. 14 is a schematic perspective view illustrating a portion of another pressure sensor according to the embodiment.

In the sensing element 50C as shown in FIG. 14, the lower electrode 204, the foundation layer 205, the pinning layer 206, the first fixed magnetic layer 209, the intermediate layer 203, the free magnetic layer 210, and the capping layer 211 are stacked in this order. For example, the sensing element 50C has a single pinned structure that uses a single fixed magnetic layer.

The foundation layer 205 includes, for example, Ta/Ru. The thickness (the length in the Z-axis direction) of the Ta layer is, for example, 3 nm. The thickness of the Ru layer is, for example, 2 nm. The pinning layer 206 includes, for example, an IrMn-layer having a thickness of 7 nm. The first fixed magnetic layer 209 includes, for example, a Co₄₀Fe₄₀B₂₀ layer having a thickness of 3 nm. The intermediate layer 203 includes, for example, a MgO layer having a thickness of 1.6 nm. The free magnetic layer 210 includes, for example, Co₄₀Fe₄₀B₂₀ having a thickness of 4 nm. The capping layer 211 includes, for example, Ta/Ru. The thickness of the Ta layer is, for example, 1 nm. The thickness of the Ru layer is, for example, 5 nm.

For example, materials similar to the materials of the layers of the sensing element 50A are used as the materials of the layers of the sensing element 50C.

FIG. 15 is a schematic perspective view illustrating a portion of another pressure sensor according to the embodiment.

In the sensing element 500 as shown in FIG. 15, the lower electrode 204, the foundation layer 205, a lower pinning layer 221, a lower second fixed magnetic layer 222, a lower magnetic coupling layer 223, a lower first fixed magnetic layer 224, a lower intermediate layer 225, a free magnetic layer 226, an upper intermediate layer 227, an upper first fixed magnetic layer 228, an upper magnetic coupling layer 229, an upper second fixed magnetic layer 230, an upper pinning layer 231, and the capping layer 211 are stacked in order.

The foundation layer 205 includes, for example, Ta/Ru. The thickness (the length in the Z-axis direction) of the Ta layer is, for example, 3 nanometers (nm). The thickness of the Ru layer is, for example, 2 nm. The lower pinning layer 221 includes, for example, an IrMn-layer having a thickness of 7 nm. The lower second fixed magnetic layer 222 includes, for example, a Co₇₅Fe₂₅ layer having a thickness of 2.5 nm. The lower magnetic coupling layer 223 includes, for example, a Ru layer having a thickness of 0.9 nm. The lower first fixed magnetic layer 224 includes, for example, a Co₄₀Fe₄₀B₂₀ layer having a thickness of 3 nm. The lower intermediate layer 225 includes, for example, a MgO layer having a thickness of 1.6 nm. The free magnetic layer 226 includes, for example, Co₄₀Fe₄₀ having a thickness of 4 nm. The upper intermediate layer 227 includes, for example, a MgO layer having a thickness of 1.6 nm, The upper first fixed magnetic layer 228 includes, for example, Co₄₀Fe₄₉B₂₀/Fe₅₀Co₅₀. The thickness of the Co₄₀Fe₄₀B₂₀ layer is, for example, 2 nm. The thickness of the Fe₅₀Co₅₀ layer is, for example, 1 nm. The upper magnetic coupling layer 229 includes, for example, a Ru layer having a thickness of 0.9 nm. The upper second fixed magnetic layer 230 includes for example, a Co₇₅Fe₂₅ layer having a thickness of 2.5 nm. The upper pinning layer 231 includes, for example, an IrMn-layer having a thickness of 7 nm. The capping layer 211 includes, for example, Ta/Ru. The thickness of the Ta layer is, for example, 1 nm. The thickness of the Ru layer is, for example, 5 nm.

For example, materials similar to the materials of the layers of the sensing element 50A are used as the materials of the layers of the sensing element 50D.

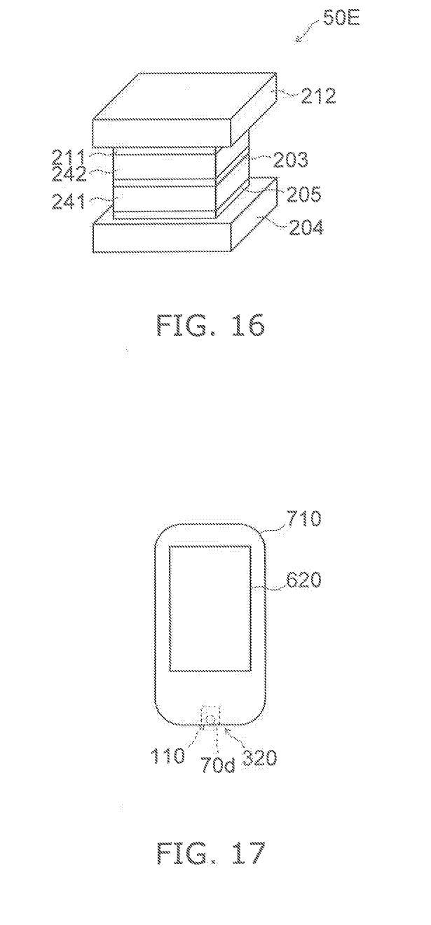

FIG. 16 is a schematic perspective view illustrating a portion of another pressure sensor according to the embodiment.

In the sensing element 50E as shown in FIG. 16, the lower electrode 204, the foundation layer 205, a first free magnetic layer 241, the intermediate layer 203, a second free magnetic layer 242, the capping layer 211, and the upper electrode 212 are stacked in this order. In the example, the magnetizations of the first opposing magnetic layer 11b and the second opposing magnetic layer 12b are changeable.

The foundation layer 205 includes, for example, Ta/Cu. The thickness (the length in the Z-axis direction) of the Ta layer is, for example, 3 nm. The thickness of the Cu layer is, for example, 5 nm. The first free magnetic layer 241 includes, for example, Co₄₀Fe₄₀B₂₀ having a thickness of 4 nm. The intermediate layer 203 includes, for example, Co₄₀Fe₄₀B₂₀ haying a thickness of 4 nm. The capping layer 211 includes, for example, Cu/Ta/Ru. The thickness of the Cu layer is, for example, 5 nm. The thickness of the Ta layer is, for example, 1 nm. The thickness of the Ru layer is, for example, 5 nm.

Materials similar to the materials of the layers of the sensing element 50A are used as the materials of the layers of the sensing element 50E. For example, materials similar to those of the free magnetic layer 210 of the sensing element 50A may be used as the materials of the first free magnetic layer 241 and the second free magnetic layer 242.

### Second Embodiment

FIG. 17 is a schematic view illustrating an electronic device according to a second embodiment.

As shown in FIG. 17, a microphone 320 according to the embodiment includes any pressure sensor according to the embodiments recited above or a pressure sensor of a modification of the embodiments. In the example, the pressure sensor 110 is used as the pressure sensor.

For example, the microphone 320 is provided in an electronic device 710 (e.g., a personal digital assistant). For example, the film portion 70d of the pressure sensor 110 is substantially parallel to the surface in which a display portion 620 of the electronic device 710 is provided. The arrangement of the film portion 70d is arbitrary. According to the embodiment, a microphone can be provided in which the dynamic range can be enlarged. The microphone 320 according to the embodiment may be provided in, for example, an IC recorder, a pin microphone, etc.

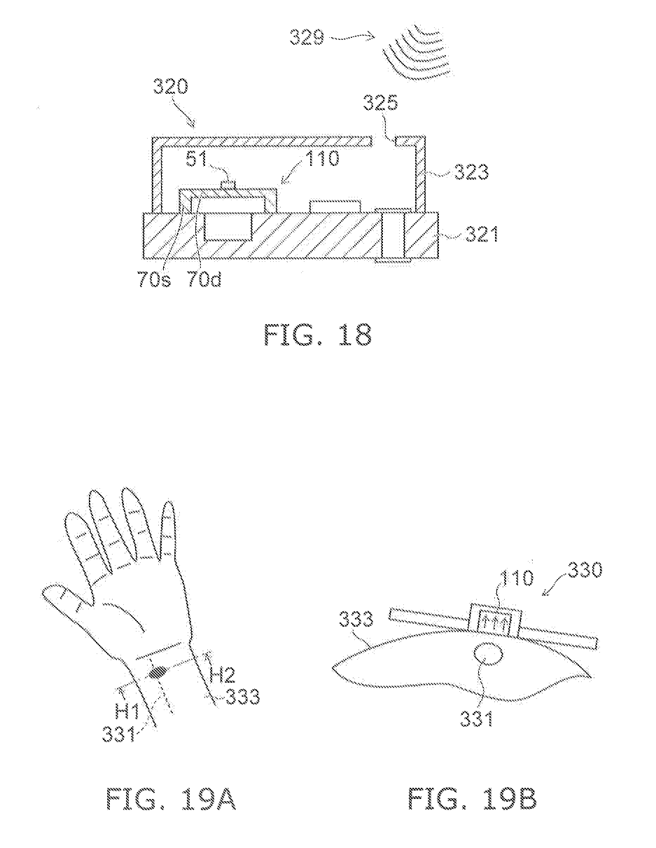

FIG. 18 is a schematic cross-sectional view illustrating the microphone according to the second embodiment.

The microphone 320 (the acoustic microphone) according to the embodiment includes a substrate 321 (e.g., a printed circuit board), a cover 323 (a housing), and a pressure sensor. Any pressure sensor according to the embodiments or a modification of any pressure sensor according to the embodiments is used as the pressure sensor. In the example, the pressure sensor 110 is used as the pressure sensor. The substrate 321 includes, for example, a circuit such as an amplifier, etc. An acoustic hole 325 is provided in the cover 323. Sound 329 passes through the acoustic hole 325 and enters the interior of the cover 323.

The microphone 320 responds to the sound pressure. For example, the pressure sensor 110 is provided on the substrate 321. An electrical signal line is provided. The cover 323 is provided on the substrate 321 to cover the pressure sensor 110. The support body 70s, the film portion 70d, the first sensing element 51, and the structure body 41 (not illustrated) are positioned between the substrate 321 and the cover 323.

### Third Embodiment

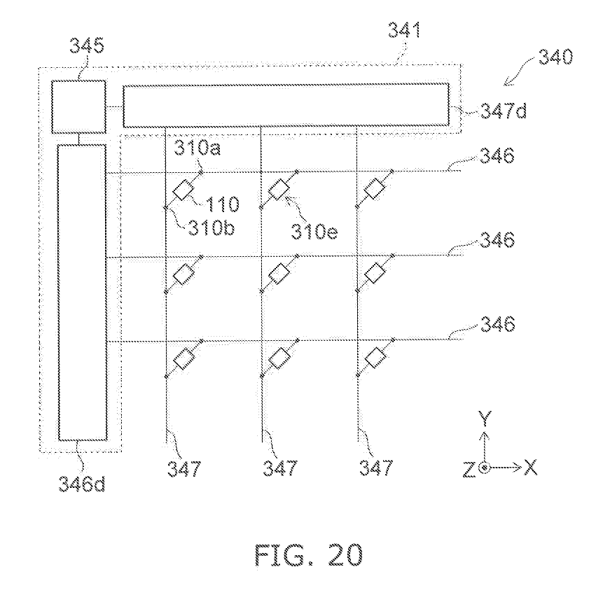

FIG. 19A and FIG. 19B are schematic views illustrating a blood pressure sensor according to a third embodiment.

FIG. 19A is a schematic plan view illustrating skin on an arterial vessel of a human. FIG. 19B is a line H1-H2 cross-sectional view of FIG. 19A.

The blood pressure sensor 330 according to the embodiment includes any pressure sensor according to the embodiments or a modification of any pressure sensor according to the embodiments. In the example, the pressure sensor 110 is used as the pressure sensor. The pressure sensor 110 is pressed onto the skin 333 on the arterial vessel 331. Thereby, the blood pressure sensor 330 can continuously perform blood pressure measurements. The blood pressure can be measured with high sensitivity. The blood pressure sensor 330 is one electronic device.

### Fourth Embodiment

FIG. 20 is a schematic view illustrating a touch panel according to a fourth embodiment.

The touch panel 340 according to the embodiment includes any pressure sensor according to the embodiments or a modification of any pressure sensor according to the embodiments. In the example, the pressure sensor 110 is used as the pressure sensor. In the touch panel 340, the pressure sensors 110 are mounted to at least one of the interior of the display or the exterior of the display. The touch panel 340 is one electronic device.

For example, the touch panel 340 includes multiple first interconnects 346, multiple second interconnects 347, the multiple pressure sensors 110, and a controller 341.

In the example, the multiple first interconnects 346 are arranged along the Y-axis direction. The multiple first interconnects 346 extend along the X-axis direction. The multiple second interconnects 347 are arranged along the X-axis direction. The multiple second interconnects 347 extend along the Y-axis direction.

One of the multiple pressure sensors 110 is provided at the crossing portion between one of the multiple first interconnects 346 and one of the multiple second interconnects 347. One of the pressure sensors 110 is used as one of sensing components 310e for sensing. Here, the crossing portion includes the position where the first interconnect 346 and the second interconnect 347 cross and includes the region at the periphery of the position.

One end 310a of one of the multiple pressure sensors 110 is connected to one of the multiple first interconnects 346. The other end 310b of the one of the multiple pressure sensors 110 is connected to one of the multiple second interconnects 347.

The controller 341 is connected to the multiple first interconnects 346 and the multiple second interconnects 347. For example, the controller 341 includes a first interconnect circuit 346d that is connected to the multiple first interconnects 346, a second interconnect circuit 347d that is connected to the multiple second interconnects 347, and a control circuit 345 that is connected to the first interconnect circuit 346d and the second interconnect circuit 347d. A high definition touch panel is obtained.

According to the embodiments, a pressure sensor and an electronic device can be provided in which the sensing characteristics can be stabilized.

In the specification of the application, “perpendicular” and “parallel” refer to not only strictly perpendicular and strictly parallel but also include, for example, the fluctuation due to manufacturing processes, etc. It is sufficient to be substantially perpendicular and substantially parallel.

Hereinabove, exemplary embodiments of the invention are described with reference to specific examples. However, the embodiments of the invention are not limited to these specific examples. For example, one skilled in the art may similarly practice the invention by appropriately selecting specific configurations of components included in pressure sensors such as film portions, sensing elements, magnetic layers, intermediate layers conductive layers structure bodies, processors, etc., from known art. Such practice is included in the scope of the invention to the extent that similar effects thereto are obtained.

Further, any two or more components of the specific examples may be combined within the extent of technical feasibility and are included in the scope of the invention to the extent that the purport of the invention is included.

Moreover, all pressure sensors and electronic devices practicable by an appropriate design modification by one skilled in the art based on the pressure sensors and electronic devices described above as embodiments of the invention also are within the scope of the invention to the extent that the purport of the invention is included. Various other variations and modifications can be conceived by those skilled in the art within the spirit of the invention, and it is understood that such variations and modifications are also encompassed the scope of the invention.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the invention.

What is claimed is:

1. A sensor, comprising:
a support body;
a film portion supported by the support body, the film portion being deformable;
a first sensing element fixed to the film portion, the first sensing element including a first magnetic layer, a first opposing magnetic layer, and a first intermediate layer, the first opposing magnetic layer being provided between the first magnetic layer and the film portion, the first intermediate layer being provided between the first magnetic layer and the first opposing magnetic layer; and
a structure body including a first region and a second region, the first region overlapping the support body in a first direction, the second region being continuous with the first region and overlapping the film portion in the first direction, the first direction being from the first opposing magnetic layer toward the first magnetic layer, the structure body including a first structure body layer, a first opposing structure body layer, and a first structure body intermediate layer, the first structure body layer including a material included in the first magnetic layer, the first opposing structure body layer including a material included in the first opposing magnetic layer and being provided between the first structure body layer and the support body and between the first structure body layer and the film portion, the first structure body intermediate layer including a material included in the first intermediate layer and being provided between the first structure body layer and the first opposing structure body layer.

2. The sensor according to claim 1, further comprising a second sensing element fixed to the film portion,
the second sensing element including:
a second magnetic layer;
a second opposing magnetic layer provided between the second magnetic layer and the film portion; and
a second intermediate layer provided between the second magnetic layer and the second opposing magnetic layer,
at least a portion of the second region being between the first sensing element and the second sensing element.

3. The sensor according to claim 2, wherein
an outer edge of the film portion includes a first side and a second side,
the first, side extends along a second direction crossing the first direction,
the second side extends along the second direction,
a third direction from the second side toward the first side crosses a plane including the first direction and the second direction,
the first sensing elements is provided in a plurality,
the second sensing elements is provided in a plurality,
the plurality of first sensing elements are arranged along the first side, and
the plurality of second sensing elements are arranged along the second side.

4. The sensor according to claim 3, wherein
the outer edge further includes a third side and a fourth side,
the third side extends along the third direction,
the fourth side extends along the third direction,
a direction from the third side toward the fourth side is aligned with the second direction, and
a length along the second direction of the first side is longer than a length along the third direction of the third side.

5. The sensor according to claim 2, further comprising a third sensing element and a fourth sensing element fixed to the film portion,
the third sensing element including:
a third magnetic layer;
a third opposing magnetic layer provided between the third magnetic layer and the film portion; and
a third intermediate layer provided between the third magnetic layer and the third opposing magnetic layer,
the fourth sensing element including:
a fourth magnetic layer;
a fourth opposing magnetic layer provided between the fourth magnetic layer and the film portion; and
a fourth intermediate layer provided between the fourth magnetic layer and the fourth opposing magnetic layer,
at least a portion of the second region being between the third sensing element and the fourth sensing element.

6. The sensor according to claim 5, wherein
an outer edge of the film portion includes a first side, a second side, a third side, and a fourth side,
the first side extends along a second direction crossing the first direction,
the second side extends along the second direction,
a third direction from the second side toward the first side crosses a plane including the first direction and the second direction,
the third side extends along the third direction,
the fourth side extends along the third direction,
a direction from the third side toward the fourth side is aligned with the second direction,
the first sensing elements is provided in a plurality,
the second sensing elements is provided in a plurality,
the third sensing elements is provided in a plurality,
the fourth sensing elements is provided in a plurality,
the plurality of first sensing elements are arranged along the first side,
the plurality of second sensing elements are arranged along the second side,
the plurality of third sensing elements are arranged along the third side and
the plurality of fourth sensing elements are arranged along the fourth side.

7. The sensor according to claim 6, wherein
the first sensing elements are connected in series to each other,
the second sensing elements are connected in series to each other,
the third sensing elements are connected in series to each other,
the fourth sensing elements are connected in series to each other,
one end of the first sensing elements connected in series is electrically connected to a first node of a bridge circuit,
another end of the first sensing elements connected in series is electrically connected to a second node of the bridge circuit,
one end of the third sensing elements connected in series is electrically connected to the second node,
another end of the third sensing elements connected in series is electrically connected to a third node of the bridge circuit,
one end of the second sensing elements connected in series is electrically connected to the third node,
another end of the second sensing elements connected in series is electrically connected to a fourth node of the bridge circuit,
one end of the fourth sensing elements connected in series is electrically connected to the fourth node, and
another end of the fourth sensing elements connected in series is electrically connected to the first node.

8. The sensor according to claim 1, wherein
the structure body further includes a third region, a fourth region, and a fifth region continuous with the second region,
the third region, the fourth region, and the fifth region overlap the support body in the first direction,
at least a portion of the first sensing element is between the first region and the third region in a direction crossing the first direction,
at least a portion of the second sensing element is between the fourth region and the fifth region in a direction crossing the first direction,
at least a portion of the third sensing element is between the first region and the fourth region in a direction crossing the first direction, and
at least a portion of the fourth sensing element is between the third region and the fifth region in a direction crossing the first direction.

9. The sensor according to claim 1, wherein
the structure body further includes a third region continuous with the second region,
the third region overlaps the support body in the first direction, and
at least a portion of the second region is positioned between the first region and the third region.

10. The sensor according to claim 1, wherein at least a portion of the first sensing element overlaps the first region in a direction crossing the first direction.

11. The sensor according to claim 1, wherein a portion of the film portion is separated from the support body.

12. The sensor according to claim 1, wherein the film portion includes a portion not overlapping the structure body in the first direction.

13. The sensor according to claim 2, wherein
the film portion includes:
a portion overlapping the structure body in the first direction; and
a portion not overlapping the structure body in the first direction,
the overlapping portion is provided around the non-overlapping portion, and
the non-overlapping portion extends along a direction from the second sensing element toward the first sensing element.

14. The sensor according to claim 1, wherein
the first sensing element further includes a first conductive layer and a second conductive layer,
the first magnetic layer is positioned between the first conductive layer and the second conductive layer,
the first opposing magnetic layer is positioned between the second conductive layer and the first magnetic layer,
the structure body further includes a first structure body conductive layer and a second structure body conductive layer,
the first structure body layer is positioned between the first structure body conductive layer and the second structure body conductive layer,
the first opposing structure body layer is positioned between the second structure body conductive layer and the first structure body layer,
the first structure body conductive layer includes a material included in the first conductive layer, and
the second structure body conductive layer includes a material included in the second conductive layer.

15. The sensor according to claim 1, wherein an electrical resistance of the first sensing element changes according to a deformation of the film portion.

16. The sensor according to claim 1, wherein
the first structure body layer is insulated from the first magnetic layer and the first opposing magnetic layer, and
the first opposing structure body layer is insulated from the first magnetic layer and the first opposing magnetic layer.

17. The sensor according to claim 1, wherein the first structure body layer is electrically connected to the first opposing structure body layer.

18. The sensor according to claim 1, further comprising:
a substrate; and
a cover,
the support body, the film portion, the first sensing element, and the structure body being positioned between the substrate and the cover.

19. A sensor, comprising
a support body;
a film portion supported by the support body, the film portion being deformable;
a first sensing element fixed to the film portion, the first sensing element including a first magnetic layer; and
a structure body including a first region and a second region, the first region overlapping the support body, the second region being continuous with the first region and overlapping the film portion, the structure body including a first structure body layer, including a material included in the first magnetic layer.

20. An electronic device, comprising:
the sensor according to claim 1, and
a housing.
